(19) United States
(12) Patent Application Publication
(10) Pub. No.: US 2019/0321576 A1
(43) Pub. Date: Oct. 24, 2019

(54) GAS MIXER FOR PROVING A GAS MIXTURE TO A MECHANICAL VENTILATOR

(71) Applicant: Air Liquide Santé (International), Paris (FR)

(72) Inventor: Thierry BOULANGER, Media, PA (US)

(21) Appl. No.: 16/262,013

(22) Filed: Jan. 30, 2019

Related U.S. Application Data
(60) Provisional application No. 62/660,303, filed on Apr. 20, 2018.

(51) Int. Cl. A61M 16/12; A61M 16/10; A61M 16/20

(52) U.S. Cl. CPC A61M 16/122; A61M 2016/0027; A61M 16/203; A61M 16/104

(57) ABSTRACT

The invention concerns a gas mixer (1) that is adapted for providing a gas mixture to a mechanical ventilator, comprising a mixing vessel (53), a first line (10) for providing a first gas, a second line (20) for providing a second gas and a third line (30) for providing a third gas, said first, second and third lines (10, 20, 30) being in fluid communication with the mixing vessel (53) for proving said first, second and third gases to said mixing vessel (53) and obtaining a gas mixture in said mixing vessel (53), and a delivery line (60) in fluid communication with the mixing vessel (53) for recovering at least a part of the gas mixture contained in the mixing vessel (53).

### CROSS REFERENCE TO RELATED APPLICATIONS

This application claims the benefit of priority under 35 U.S.C. § 119 (a) and (b) to U.S. Provisional Application No. 62/660,303, filed Apr. 20, 2018, the entire contents of which are incorporated herein by reference.

### BACKGROUND

The present invention relates to a gas mixing device or mixer that can be used in hospitals for operating precise deliveries of various gas mixtures, especially when fluidly connected to a medical ventilator.

Gas mixtures are commonly used for treating various respiratory deficiencies or diseases affecting different populations of patients.

For instance, oxygen-enriched air is often used for treating hypoxemic conditions. Such an oxygen/air mixture can be obtained in situ, typically directly in the nostrils of the patient, by mixing an O₂ flow delivered by a nasal cannula, at a continuous flowrate of between 1 and 6 L/min, with ambient air inspired by the patient during inspiratory phases.

In case of a severe respiratory distress, such as COPD exacerbation affecting adult patients or acute asthma attacks affecting pediatric patients, at least a part of the nitrogen molecules (N₂) contained in air can be replaced by helium (He) to decrease the breathing work of the patients. This is typically done by means of a gas mixing device, also called “mixer” or “blender”, which is connected to several sources of gas, such as O₂ and He. The O₂ concentration, i.e. the He/O₂ ratio, is generally adjusted by the medical staff as it depends on the patient's needs.

Medical gases are also used as therapeutic agents for treating other medical conditions or diseases. For instance, pain and anxiety can be relieved by inhaling a gaseous mixture of O₂ and nitrous oxide (N₂O), whereas a ternary mixture containing 50 vol. % argon (Ar), 21 vol. % O₂ and 29% N₂ (vol. %) has shown neuroprotective properties.

Despite the fact that gas mixtures are widely used for treating patients, especially in hospitals or the like, delivering a hypoxic mixture to the patient is a constant risk and it is important to make sure the concentration of oxygen in the gas mixture administrated to the patients is never unintentionally less than 21 vol. %.

The gas administration has hence to be controlled both in operation and in case of failure of the gas mixing device.

Architectures of gas mixers have been proposed by US-A-2008/0121233, US-A-2009/0205661 and EP-A-2489392.

For instance, EP-A-2489392 discloses a gas mixing device (blender) including a pressure vessel of about 0.5 L which stores the gas mixture above the atmospheric pressure, for example at 2 bar abs. An on-demand valve is arranged between the pressure vessel and the patient. This valve opens only when a pressure decrease occurs (due to an inhalation phase of the patient) and a part of the gas mixture of the vessel is then delivered to the patient. The vessel is refilled when the gas pressure measured in said vessel drops below a given threshold value.

However, with existing blenders, it can be difficult to realize a precise control of the concentrations in the gas mixture, especially the oxygen concentration, and further to detect hypoxic situations that could be risky for the patient.

SUMMARY

A goal of the present invention is to provide a gas mixer able to deliver a concentration of up to three gases, such as a therapeutic gas, oxygen and air, that overcomes at least part of the drawbacks of the blenders of the prior art.

A solution according to the present invention concerns a gas mixer comprising:

a mixing vessel,

a first line for providing a first gas, a second line for providing a second gas and a third line for providing a third gas, said first, second and third lines being in fluid communication with the mixing vessel for proving said first, second and third gases to said mixing vessel and obtaining a gas mixture in said mixing vessel, and

a delivery line in fluid communication with the mixing vessel for recovering at least a part of the gas mixture contained in the mixing vessel,

and wherein:

the first line comprises a first pressure regulator, a first pressure sensor and a first mass flow controller comprising a first proportional valve and a first mass flow sensor,

the second line comprises a second pressure regulator, a second pressure sensor and a second mass flow controller comprising a second proportional valve and a second mass flow sensor,

the third line comprises a third pressure regulator, a third pressure sensor and a third mass flow controller comprising a third proportional valve and a third mass flow sensor,

the first, the second and the third lines are in fluid communication with the mixing vessel via an admission line, and

an oxygen sensor is arranged on the admission line.

Depending on the embodiment, a gas mixer according to the present invention can comprise one or several of the following features:

the admission line is in fluid communication with the first, second and third lines and with the mixing vessel.

the admission line is in fluid communication with the second and third lines via a first common line.

a fourth mass flow sensor is arranged on the first common line.

a fifth mass flow sensor is arranged on the admission line between the oxygen sensor and the mixing vessel.

the mixing vessel is further fluidly connected to a delivery line for withdrawing and conveying at least a part of the gaseous mixture contained in said mixing vessel.

the delivery line comprises a fourth proportional valve.

the delivery line further comprises a fourth pressure sensor arranged downstream of the fourth proportional valve.

the mixer further comprises a control unit.

the first line is fluidly connected to a first gas source, the second line is fluidly connected to a second gas source and the third line is fluidly connected to a third gas source.

the first gas source delivers air.

the second gas source delivers oxygen gas.

the third gas source delivers a therapeutically-effective gas.

the therapeutically-effective gas is chosen among N₂O, argon, xenon, helium or nitrogen.

the mixer comprises a setting interface connected to the control unit.

the control unit is configured for processing pressure signals received from the pressure sensors and from the mass flow sensors.

the control unit is further configured for processing oxygen concentration signals received from the oxygen sensor.

the control unit is further configured for controlling the proportional valves.

Further, the invention also concerns a mechanical ventilation system comprising gas mixer according to the present invention, in fluid communication with a mechanical ventilator for providing a gas mixture to said mechanical ventilator.

### BRIEF DESCRIPTION OF THE DRAWINGS

The present invention will be explained in more details in the following illustrative description of an embodiment of a gas mixing device or mixer according to the present invention, which is made in references to the accompanying drawings among them:

FIG. 1 shows an embodiment of the architecture of a gas mixer according to the present invention,

FIG. 2 shows a gas mixer cooperating with a mechanical ventilator, and

FIG. 3 shows a second embodiment of a mixer according to the present invention that is able to operate with a mechanical ventilator.

### DESCRIPTION OF THE PREFERRED EMBODIMENTS

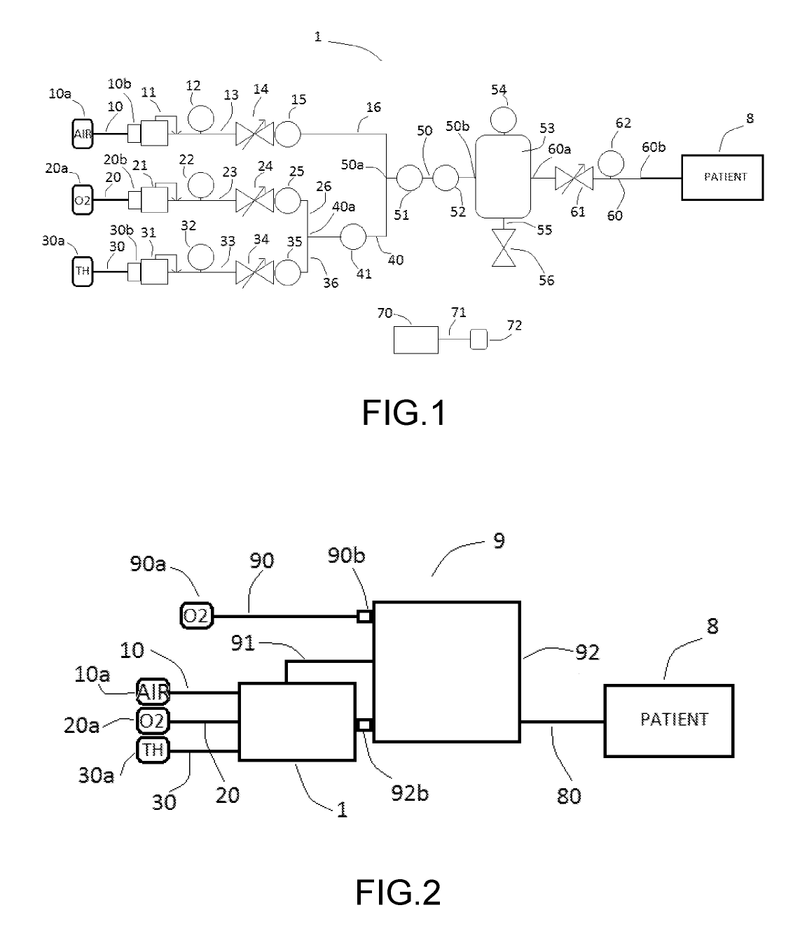

In FIG. 1, a first embodiment of a gas mixer 1 according to the present invention, that is fluidly connected to a patient 8 at the end 60b of a delivery line 60, is shown.

The mixer 1 of FIG. 1 comprises a first, a second and a third gas line 13, 23, 33, respectively, comprising a first, a second and a third inlet port 10b, 20b, 30b, respectively, wherein:

the first inlet port 10b is fluidly connected, via a first inlet line 10, to a first source 10a of a first gas, namely a medicinal air source, for instance an air delivery plug arranged on a wall and fed by the gas network of a hospital,

the second port 20b is fluidly connected, via a second inlet line 20, to a second source 20a of a second gas, namely a medical O₂ source 20a, for instance an oxygen delivery plug also fed by the gas network of the hospital,

the third port 30b is fluidly connected, via a third inlet line 30, to a third source 30a of a third gas, namely a therapeutically-effective gas (TH) source 30a, preferably a gas cylinder or the like. For instance, the therapeutically-effective gas can be N₂O, argon, xenon, helium or nitrogen.

The first, second and third gas lines 13, 23, 33 are arranged in parallel.

The mixer 1 comprises different electronic elements, such as mass flow controllers and sensors, that are controlled by a control unit 70, whose core component is a microprocessor or a microcontroller.

The control unit 70 further provides electric power to the electronic elements and is able to read and process the measurements provided by the sensors, typically signals delivered by the sensors.

The control unit 70 embeds conversion lookup tables or the like, for determining a flow measurement based on the nature of the gas being measured, here for instance medicinal air, 02, therapeutic gas and combinations thereof.

The control unit 70 is further in communication, via electric line 71, to a command unit 72 operable by a user for setting the concentrations of the different gases in the mixture to be realized in vessel 53, which is afterwards delivered to the patient 8 via delivery line 60.

First line 13 comprises, in series, downstream of the first inlet port 10b, a first pressure regulator 11, a first pressure sensor 12 and a first mass flow controller 14, 15. The role of each of those elements is detailed hereafter.

First pressure regulator 11 is provided for reducing the pressure of the first gas, e.g. air, delivered by the first gas source, e.g. a medicinal air source 10a. The level of the desired lower pressure depends on the settings of the first pressure regulator 11. For instance, if the pressure of the first gas is of about 3.5 bars abs, the pressure level of the first pressure regulator 11 can be set to a lower pressure of 2.5 bars abs (of course, it could be higher or lower) so that the first gas exhibits said lower pressure downstream of the first pressure regulator 11, in first line 13.

First pressure sensor 12, that is in communication with control unit 70, is used for monitoring the gas pressure in first line 13, downstream of the first pressure regulator 11, to make sure that the pressure value of the first gas is within acceptable ranges, for instance +/− %5 with respect to a given pressure value. First pressure sensor 12 can also indicate to control unit 70 that no or insufficient pressure exists in first line 13, which means that the first source 10a, such as a medicinal air source, is not connected to the first inlet port 10b.

First mass flow controller 14, 15 comprises a first proportional valve 14 and a first mass flow sensor 15. The first proportional valve 14 is controlled by control unit 70 which determines its opening status (i.e. valve more or less open) based on a targeted flow measured by the first mass flow sensor 15, whereas first mass flow sensor 15 is configured by a lookup table relating to the first gas, such as medicinal air. The gaseous flow passing through first proportional valve 14 travels first into first mass flow sensor 15 and then in the downstream portion 16 of first line 13.

Similarly, the second line 23 comprises, in series, downstream of the second inlet port 20b receiving the second gas from the second source 20a, e.g. a medicinal O₂ source, a second pressure regulator 21, a second pressure sensor 22 and a second mass flow controller 24, 25 comprising a second proportional valve 24 and a second mass flow sensor 25, whereas the third line 33 also comprises, in series, downstream of the third inlet port 30b receiving the third gas from the third source 30a, e.g. a therapeutically-effective gas source, a third pressure regulator 31, a third pressure sensor 32 and a third mass flow controller 34, 35 comprising a third proportional valve 34 and a third mass flow sensor 35. As all those elements run and/or are operated the same way as the corresponding ones arranged on the first line 13, they will not be detailed further.

The mixer 1 provides the ability to deliver up to 3 gases coming from the first, second and third gas sources 10a, 20a, 30a.

As the first, second and third pressure sensors 12, 22 and 32 inform the control unit 70 of the presence of gas sources fluidly connected to the first, second and third ports 10b, 20b, 30b, some specific gas mixtures can be prohibited for increasing the safety for the patient 8. This means that the control unit 70 can be configured for not authorizing the delivery of some specific mixtures to patient 8.

For instance, control unit 70 can be configured for

allowing the delivery of gas if it is detected that second gas source 20a (e.g. medical O₂) and third gas source 30a (e.g. therapeutic gas) and/or first gas source 10a (e.g. medicinal air) are simultaneously connected to their respective ports 10b, 20b, 30b, or

in contrast, prohibiting the delivery of gas if it is detected that the third gas source 30a (e.g. therapeutic gas) is the only source connected or that the first gas source 10a (e.g. medicinal air) is the only additional source simultaneously connected with third gas source 30a.

In other words, thanks to the control unit 70 that independently pilots the first, second and third proportional valves 14, 24, 34, the mixer 1 of the present invention is able to deliver a mixture of only two gases, for instance a mixture of 50 vol. % O₂ and 50 vol. % N₂O as the therapeutic gas, or three gases, for instance a mixture of 50 vol. % Ar as the therapeutic gas, 21% O₂ and the rest being N₂; of course, other amounts of gas can be delivered.

As shown in FIG. 1, the downstream portions 26, 36 of second and third gas lines 23, 33 are fluidly connected to a first common line 40, i.e. they are branched at a first location 40a, so that gases coming from the second and third gas lines 23, 33 can travel in said first common line 40 toward vessel 53.

In the same way, common line 40 and downstream portion 16 of first gas line 13 are fluidly connected to a second common line 50, also called admission line, i.e. they are branched at second location 50a, so that gases coming from the first gas line 13 and from the first common line 40 (that receives gas from the second and third gas lines 23, 33) can travel in said admission line 50, toward vessel 53.

Admission line 50 or second common line is fluidly connected to pressure vessel 53 via an inlet orifice 50b, for delivering the different gases to be mixed in said vessel 53.

The pressurized vessel 53 has a suitable volume typically comprised between 0.1 and 5 L, for example a volume of about 0.5 L, wherein the gas mixture is stored at a desired pressure or in a desired pressure range, for instance between 2 and 2.5 bars abs. Vessel 53 constitutes a mixing chamber for the gases.

Mixing vessel 53 is further fluidly connected to the upstream end 60a of a delivery line 60 which conveys the gaseous mixture withdrawn from vessel 53 to the patient 8. Delivery line 60 comprises a fourth proportional valve 61 and a fourth pressure sensor 62, the latter being arranged downstream of the fourth proportional valve 61.

Control unit 70 receives pressure measurement signals from the fourth pressure sensor 62 and further controls said fourth proportional valve 61 based on said pressure measurement signals.

The downstream portion 60b of delivery line 60 delivers the gaseous mixture to the patient 8, for instance by means of a respiratory interface, such as a respiratory mask or the like.

When a patient 8 is connected to terminal downstream end 60b of delivery line 60 and starts to inhale gas, the pressure downstream of proportional valve 61 falls below the atmospheric pressure, which will be measured/detected by the fourth pressure sensor 62.

In response to that pressure decreasing, control unit 70 that controls the fourth proportional valve 61 sets the opening status of the fourth proportional valve 61 to counterbalance the pressure decreasing measured by the fourth pressure sensor 62, that reflects the patient's flow demand. A part of the gas mixture stored in vessel 53 is then drawn into delivery line 60, whereas the pressure in vessel 53 decreases. Said pressure decreasing is detected by vessel pressure sensor 54 that is arranged on vessel 53 so as to monitor the pressure variations in pressure vessel 53.

Measurement signals delivered by vessel pressure sensor 54 are recovered by control unit 70 that is also electrically connected to vessel pressure sensor 54.

In response to that pressure decrease in vessel 53, control unit 70 actuates first, second and third proportional valves 14, 24, 34 so as to allow a passage of the different gases through said valves 14, 24, 34. The pressurized gases travel in the different lines 16, 26, 36, 40 and admission line 50 before reaching vessel 53 thereby refilling vessel 53 with the right gas mixture until the pressure in said vessel 53, measured by vessel pressure sensor 54, reaches again the desired pressure range, for instance between 2 and 2.5 bars abs.

According to the present invention, it is also provided safety elements for preventing hypoxic situations, precisely controlling the gas mixture delivered to the patient 8 and monitoring the concentration of the gas mixture realized in vessel 53.

More precisely, according to the present invention, it is operated a pre-use verification sequence and subsequently a real-time monitoring of the mixer status to guarantee a safe and precise operation.

### Pre-Use Verification Sequence

The pre-use verification sequence is initiated by the control unit 70 in response to a command input by the operator operated on the command unit 72 that is connected to the control unit 70 by means of a connecting line 71.

Upon initiation of the pre-use verification, control unit 70 shuts the first, second and third proportional valves 14, 24 and 34 so that no gas can travel in the first, second and third lines 16, 26 and 36.

Meanwhile, control unit 70 shuts the fourth proportional valve 61 to prevent gas travel into delivery line 60, in the case where a patient 8 is already connected to line 60, and opens a dump valve 56 which is in fluid communication with the inner volume of pressure vessel 53, via a venting line 55. Dump valve 56, when opened, allows the gas contained into vessel 53 to be vented to the atmosphere.

After this sequence is performed, control unit 70 opens the third proportional valve 34 so that a flow of about 1 L/min, measured by the third mass flow sensor 35 travels into third line 36 and first common line 40. Line 40 comprises a fourth mass flow sensor 41.

At this stage, control unit 70 configures the fourth mass flow sensor 41 with a lookup table corresponding to the first gas source 30a containing the therapeutically-effective gas.

A first verification step made by control unit 70 comprises a step of confirming that third and fourth mass flow sensors 35, 41 measure about the same flow of gas, which significates that those third and fourth mass flow sensors 35, 41 are operational.

In admission line 50, the flow of gas then travels into two additional sensors comprising an oxygen sensor 51 and a fifth mass flow sensor 52.

Oxygen sensor 51 is for instance a zirconia based sensor, such as the one referenced KGZ-10 commercialized by the Honeywell Company, which is designed for operating at pressures up to 3 bar abs. Such a sensor is linear while operating, meaning that its output is linearly dependent on the oxygen concentration (i.e. from 0 to 100%).

At this stage, the flow of gas in contact with the oxygen sensor 51 comes from the therapeutic gas source 30a which contains 0% of O₂. This characteristic helps set a first calibration point for the oxygen sensor 51 by measuring the output of said sensor 51 via the control unit 70 and determine that said output corresponds to a 0% point (also called zeroing point).

In a same way, the fifth mass-flow sensor 52, configured by control unit 70 with respect to the properties of therapeutic gas source 30a, measures a flow equal to those measured by mass flow sensors 35, 41, further confirming its correct operation.

The flow of gas exiting admission line 50 enters vessel 53 and is vented to the atmosphere via dump valve 56 as above explained.

Once this sequence completed, the control unit 70 switches the gas sources. First proportional valve 14 is kept in its close position and third proportional valve 34 is closed, whereas second proportional valve 24 is opened so that a flow of O₂ of about 1 L/min, measured by the second mass flow sensor 25 is traveling successively into second line 26 and common line 40 that comprises the fourth mass flow sensor 41.

At this stage, control unit 70 configures the fourth mass flow sensor 41 with a lookup table corresponding to the second gas, typically medical O₂ gas, coming from the second gas source 20a.

As already explained, it is operated another verification step for confirming that the third and fourth mass flow sensors 35, 41 measure about the same flow of oxygen, for ensuring that the second mass flow sensor 35 is also operational.

The flow of oxygen travels into admission line 50, where it encounters oxygen sensor 51 and the fifth mass flow sensor 52. The flow in contact with the oxygen sensor 51 is the second gas, namely O₂ gas (purity of at least 99.5 vol. %). This leads to a second calibration point for the oxygen sensor 51 by measuring the output of said sensor 51 via control unit 70 as said output corresponds to a 100% point (also called full scale point).

As the control unit 70 has determined the 0% and the 100% of O₂ points, thanks to the calibration steps, it is able to precisely evaluate the oxygen concentration in contact with the oxygen sensor 51 by applying a linear relationship between the output of said sensor 51 and the actual content of a given gas mixture.

The oxygen flow can then enter into vessel 53 and be vented to the atmosphere via dump valve 56 as already explained.

At this stage, control unit 70 of mixer 1 has determined that second, third, fourth and fifth mass flow sensors 25, 35, 41, 52 are correctly operating, and that oxygen sensor 52 is fully calibrated.

The last step of verification consists in closing the second and third proportional valves 24, 34 and opening the first proportional valve 14 so that a flow of 1 L/min, measured by the first mass flow sensor 15, can travel into the first line 16.

Then, when proceeding as above explained for the other gases, control unit 70 is able to determine that the oxygen concentration of the first gas delivered by the first gas source 10a, namely medicinal air, contains about between 19.5% and 23.5%, of oxygen (according to the US Pharmacopoeia).

The output of oxygen sensor 51 is then stored by control unit 70. Configuring first and fifth mass flow sensors 15, 52 with a lookup table corresponding to medicinal air (the variation of the oxygen concentration into nitrogen has no impact on the measurement for mass flow sensors), it can be determined that the first mass flow sensor 15 operates correctly. Again, the flow travels to vessel 53 and is afterwards vented by dump valve 56.

This last sequence closes the pre-use verification sequence and, unless a discrepancy is found, for example mass flow sensors measurements differences, the mixer 1 is considered as being ready for operation.

### Real-Time Monitoring

Operation of mixer 1 starts when patient 8 is connected to delivery line 60 and the operator has set a gaseous mixture to be delivered to said patient 8, by means the command unit 72, such as a keyboard or similar.

As an example, a ternary mixture consisting of 50 vol. % Ar (third source 30a), 21 vol. % of O₂ (second source 20a) and 29 vol. % of air containing about 80 vol. % of N₂ (first source 10a) is selected.

As described above, the refilling of vessel 53 depends on the patient 8 demand based on the measurement of vessel pressure sensor 54 (to keep the pressure in said vessel 53 within an acceptable range, such as between 2 and 2.5 bar abs).

Whenever there is a need to refill vessel 53, control unit 70 sets different flow targets to the first, second and third proportional valves 14, 24, 34 based on the measurement of the first, second and third mass flow sensors 15, 25 and 35, which are naturally configured through proper lookup tables with respect to the gas source they are connected to.

The gaseous flows traveling in each of the first, second and third lines 16, 26, 36 are a directly related to the concentrations set by the user. Assuming for instance a total flow QTOT traveling into admission line 50 to refill vessel 53, it can be determined that flows QAR (travelling into third line 36), Q_{O2} (travelling into second line 26) and QAIR (travelling into first line 16) are as follows:

where:

Q_{AR}=C_{AR}·Q_{TOT}

Q_{AIR}=(1−C_{AR}−C_{O2})/(1−C_{O2AIR})·Q_{TOT}

Q_{O2}=Q_{TOT}−Q_{AIR}−Q_{AR}

C_{AR} is the concentration of Ar in the mixture (here 50%),

CO₂ is the concentration of O₂ in the mixture (here 21%), and

C_{O2AIR} is the concentration of O₂ in the medicinal air source (i.e. between 19.5 and 23.5 vol. %).

If C_{O2AIR} is 21%, then for a total flow Q_{TOT} of 10 L/min, Q_{AR}=5 L/min, Q_{O2}=1.33 L/min and Q_{AIR}=3.67 L/min. The gaseous flow travelling into first common line 40, which is the sum of the flows in the second and third lines 26 (Q_{O2}), 36 (Q_{AR}), is such that the oxygen concentration in said line 40 is: Q_{O2}/(Q_{AIR}+Q_{O2})=21 vol. %.

However, if C_{O2AIR} is 19.5%, Q_{AIR}=3.6 L/min and Q_{O2}=1.4 L/min, then the oxygen concentration in line 40 is of 21.8 vol. %.

Conversely, if C_{O2AIR} is 23.5%, Q_{AIR}=3.8 L/min and Q_{O2}=1.2 L/min, then the oxygen concentration in line 40 is of 19.5%.

As can be seen, taking into account the concentration of oxygen C_{O2AIR} composing the medicinal air delivered by the first source 10a, impacts the flow traveling into the downstream portion 26 of the second line 23 (i.e. medical O₂ from second source 20a) so that the overall oxygen concentration of the flow travelling into admission line 50 is at 21 vol. %.

The total flow Q_{TOT} could range between 0 to about 150 L/min (i.e. 10 L/min is an example). Consequently, the first, second and third proportional valves 14, 24, 34 are sized in a way they can also accommodate such flow ranges.

Hence, according to the present invention, for correctly operating mixer 1 delivering a precise mixture to a patient 8, it is mandatory to detect, in real time, whether the different elements, especially the mass flow sensors, operate correctly, for thereby preventing a delivery of a hypoxic mixture or a wrong gaseous mixture that does not fit with the one set by the user.

Indeed, the flow travelling into first common line 40 is the sum of the flows coming from the second line 26 (i.e. medical O₂), measured by the second mass flow sensor 25, and the flow traveling into the third line 36 (i.e. therapeutic gas), measured by the third mass flow sensor 35.

The ratio of these 2 gases yields to a specific lookup table determined by control unit 70 so that the fourth mass flow sensor 41 is correctly configured.

It should be clear at this stage that the mass flow sensor 41 should measure the sum of mass flow sensor 25 and mass flow sensor 35 measurement. Fourth mass flow sensor 41 therefore performs a “checksum” which ensures the integrity of the measurements.

Similarly, the flow traveling into admission line 50 is the sum of the flow coming from common line 40 (mixture composed of the therapeutic gas and medical oxygen), measured by fourth mass flow sensor 41, and the flow coming from first line 16 (medicinal air), measured by first mass flow sensor 15.

Here again, the ratio of these 2 gases yields to a specific lookup table determined by control unit 70 so that the fifth mass flow sensor 52 is correctly configured. The fifth mass flow sensor 52 should measure the sum of the first mass flow sensor 15 and the fourth mass flow sensor 41 measurement. Fifth mass flow sensor 52 therefore also performs a “checksum” which ensures the integrity of the measurements.

As all the mass flow sensors of mixer 1 are functioning properly, e.g. giving an accurate measurement, it can be determined the concentration of the therapeutic gas in the mixture, which is basically third mass-flow sensor 35 measurement over the total flow measured by fifth mass-flow sensor 52. It can be further confirmed that the oxygen concentration in the mixture is of about 21%, as measured by oxygen sensor 51.

As detailed above, the pre-use verification sequence ensures that all the mass-flow sensors of mixer 1 work properly and that the oxygen sensor 51 is fully calibrated.

During operation, the fourth and fifth mass flow sensors 41, 52 performs a real-time checksum thereby ensuring the integrity of the gas mixture.

In case of a sudden failure of one of the mass-flow sensors, for instance the second mass-flow sensor 25, it can be determined within a few milliseconds that the checksum performed by fourth mass flow sensor 41 is not valid anymore and mixer 1 can act accordingly by warning the user and closing the third proportional valve 34 to stop the delivery of the hypoxic therapeutic gas.

Likewise, an oxygen concentration falling below a minimum threshold of 19.5% in the medicinal air, would be detected by the oxygen sensor 51 and the mixer 1 would interrupt the therapy and warn the user.

It should be noted the importance of having the oxygen sensor 51 placed in the admission line 50, i.e. upstream of the vessel 53. Indeed, the vessel 53 is a reserve of gas at the right oxygen concentration, e.g. 21%: if the oxygen concentration measured by oxygen sensor 51 drops below 19.5%, which is typically detected within 5 s with Zirconia based oxygen sensors, only a fraction of the internal volume of vessel 53 will be replaced by the “hypoxic” concentration but the overall oxygen concentration of the mixture exiting vessel 53 will be mitigated by the large amount of gas in vessel 53 at the right concentration. Should the oxygen sensor be placed downstream of vessel 53, the patient would actually inhale an “hypoxic” mixture before the detection occurs, which is not desirable.

The example illustrating the operation of mixer 1, e.g. a mixture composed of 50% Ar, 21% O₂ and balance air (i.e. mainly N₂), allows in practice the setting of a concentration of oxygen ranging from 21 to 50 vol. %, depending on the need of the patient 8 to be treated.

Such a broad range of concentration of O₂ allows using the mixer 1 of the present invention for providing gas to various populations of patients, including those that are sedated and under mechanical ventilation.

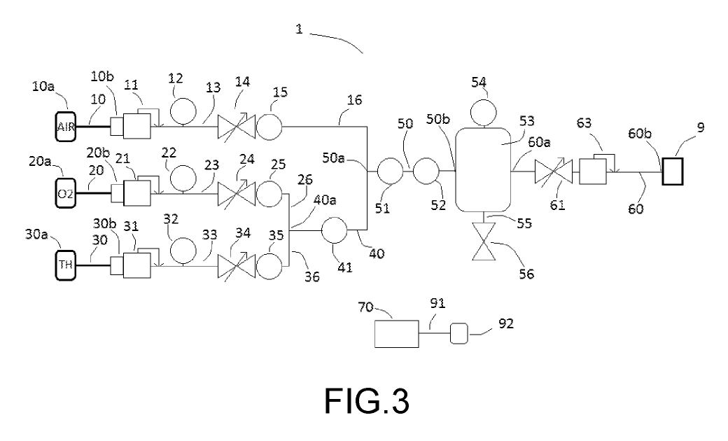

Next, FIG. 2 shows a mixer 1 according to the present invention, such as the mixer 1 of FIG. 3, connected to a mechanical ventilator 9 for feeding said ventilator 9 with a desired gas mixture and afterwards a patient 8 that is mechanically-ventilated.

The mechanical ventilator 9 delivers a life-support ventilation to the patient 8 via a breathing circuit 80, such as gas hose(s) or the like, fluidly connected to said ventilator 9.

As in FIG. 1, the mixer 1 is connected, upstream, to the first, second and third gas sources 10a, 20a, 30a via respective first, second and third inlet line 10, 20, 30, and, downstream, to an inlet port 92b of ventilator 9 so that the different gases coming from the first, second and third gas sources 10a, 20a, 30a can be mixed inside the vessel 53 contained in the mixer 1 in desired proportions, as above explained, and then delivered to the ventilator 9 as a suitable gas mixture.

In one embodiment, inlet port 92b can be, on a conventional and unmodified ventilator, the air inlet of the ventilator 9, that normally only receives medicinal air at pressure greater than 2 bar abs, whereas in another embodiment, the ventilator 9 has been modified and comprises an inlet port 92b that is specific and can only be connected to a mixer 1 according to the present invention, especially the mixer 1 of FIG. 3.

FIG. 3 shows another embodiment of a mixer 1 according to the present invention that is very similar to the one of FIG. 1, except that the pressure sensor 62 of the mixer 1 of FIG. 1 has been replaced by a fourth pressure regulator 63. The fourth pressure regulator 63 maintains a constant gas pressure in the delivery line 60, for instance a pressure of about 2 bar abs, to fit with the requirements of ventilator 9 that is fed by delivery line 60.

As shown in FIG. 2, the ventilator 9 further exhibits an additional port 90b that can only be connected via a supply line 90 to a source 90a of medical oxygen that can be the same source as the second gas source 20a, in particular when said second gas source 20a is an oxygen plug arranged on the wall of a hospital, or another oxygen source.

In operation, the ventilator 9 delivers a mechanical ventilation to the patient 8 at an oxygen concentration set by the user via a setting interface 92. The oxygen concentration can vary, depending on the patient needs, between 21 vol. % (only port 92b is used) and 50 vol. % (use of additional port 90b and gas source 90a), whereas the argon concentration in the mixture has to be kept constant, at 50 vol. %, i.e., regardless of the oxygen concentration set by the user.

Thus, when the user sets an oxygen concentration to be delivered to the patient 8:

A) set to 21% of O₂: only port 92b is opened and the mixer 1 delivers a gas mixture containing 50% Ar, 21% O₂ and 29% N₂ (vol. %),

B) set to 50% of O₂: the mixer 1 only provides a mixture of 21% O₂ and 79% Ar (no air is added as first valve 14 is closed). Supplemental oxygen is added via port 90b of the ventilator 9, thereby diluting the Ar concentration and obtaining a final mixture of 50% Ar and 50% O₂ (vol. %).

C) set to between 21% and 50% (vol. %), the mixer 1 adjusts the ratio of the different gases by controlling the first, second and third valves 14, 24, 34. Additional O₂ can be provided, if necessary, from additional O₂ source 90a.

This means that, in operation, the mixer 1 and ventilator 9 cooperate together. To this end, ventilator 9 communicates to mixer 1, via line 91 (connected to control unit 70 as shown in FIG. 3) the oxygen concentration to be delivered to the patient 8 as set by the user via the interface 92. The mixer 1 then adjusts the ratio of the gases so that a proper mixture is delivered to the ventilator 9.

Precisely controlling the concentrations of the gas mixture generated by the mixer 1, especially the oxygen concentration, is very important for ensuring a constant oxygen concentration of minimum 21% in the gas mixture delivered to the patient while keeping adequate the concentration of the therapeutic gas.

The present invention concerns a mixer 1 conceived for delivering a precisely controlled gas mixture, having the ability of monitoring in real-time the gas concentrations in said mixture and further of assessing during pre-use verification and operation, the status of its components to ensure increased safety for the patient inhaling the gas mixture delivered by said mixer 1.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

What is claimed is:

1. A gas mixer (1) comprising:
a mixing vessel (53),
a first line (10) for providing a first gas, a second line (20) for providing a second gas and a third line (30) for providing a third gas, said first, second and third lines (10, 20, 30) being in fluid communication with the mixing vessel (53) for proving said first, second and third gases to said mixing vessel (53) and obtaining a gas mixture in said mixing vessel (53), and
a delivery line (60) in fluid communication with the mixing vessel (53) for recovering at least a part of the gas mixture contained in the mixing vessel (53),
and wherein:
the first lines (10) comprises a first pressure regulator (11), a first pressure sensor (12) and a first mass flow controller (14, 15) comprising a first proportional valve (14) and a first mass flow sensor (15),
the second line (20) comprises a second pressure regulator (21), a second pressure sensor (22) and a second mass flow controller (24, 25) comprising a second proportional valve (24) and a second mass flow sensor (25),
the third line (30) comprises a third pressure regulator (31), a third pressure sensor (32) and a third mass flow controller (34, 35) comprising a third proportional valve (34) and a third mass flow sensor (35),
the first, the second and the third lines (10, 20, 30 30) are in fluid communication with the mixing vessel (53) via an admission line (50), and
an oxygen sensor (51) is arranged on the admission line (50).

2. A gas mixer (1) according to claim 1, wherein the admission line (50) is in fluid communication with the first, second and third lines (10, 20, 30 30) and with the mixing vessel (53).

3. A gas mixer (1) according to claim 1, wherein the admission line (50) is in fluid communication with the second and third lines (10, 20, 30 30) via a first common line (40).

4. A gas mixer (1) according to claim 1, wherein a fourth mass flow sensor (41) is arranged on the first common line (40).

5. A gas mixer (1) according to claim 1, wherein a fifth mass flow sensor (52) is arranged on the admission line (50) between the oxygen sensor (51) and the mixing vessel (53).

6. A gas mixer (1) according to claim 1, wherein the mixing vessel (53) is further fluidly connected to a delivery line (60) for withdrawing and conveying at least a part of the gaseous mixture contained in said mixing vessel (53).

7. A gas mixer (1) according to claim 6, wherein delivery line (60) comprises a fourth proportional valve (61).

8. A gas mixer (1) according to claim 7, wherein delivery line (60) further comprises a fourth pressure sensor (62) arranged downstream of the fourth proportional valve (61).

9. A gas mixer (1) according to claim 7, wherein delivery line (60) further comprises a fourth pressure regulator (63) arranged downstream of the fourth proportional valve (61).

10. A gas mixer (1) according to claim 1, further comprising a control unit (70).

11. A gas mixer (1) according to claim 1, wherein the first line (10) is fluidly connected to a first gas source (10a), the second line (20) is fluidly connected to a second gas source (20a) and the third line (30) is fluidly connected to a third gas source (30a).

12. A gas mixer (1) according to claim 11, wherein the first gas source (10a) is air.

13. A gas mixer (1) according to claim 11, wherein the second gas source (20a) is an oxygen gas.

14. A gas mixer (1) according to claim 11, wherein the third gas source (30a) is a therapeutically-effective gas.

15. A gas mixer (1) according to claim 14, wherein the therapeutically-effective gas is chosen among N₂O, argon, xenon, helium or nitrogen.

16. A gas mixer (1) according to claim 10, further comprising a setting interface (92) connected to the control unit (70).

17. A gas mixer (1) according to claim 10, wherein the control unit (70) is configured for processing pressure signals received from the pressure sensors (12, 22, 32) and from the mass flow sensors (15, 25, 35).

18. A gas mixer (1) according to claim 10, wherein the control unit (70) is configured for processing oxygen concentration signals received from the oxygen sensor (51).

19. A gas mixer (1) according to claim 10, wherein the control unit (70) is configured for controlling the proportional valves (14, 24, 34, 61).

20. A mechanical ventilation system (1, 9) comprising gas mixer (1) according to claim 1 in fluid communication with a mechanical ventilator (9) for providing a gas mixture to said mechanical ventilator (9).
